(19) United States
(12) Patent Application Publication
(10) Pub. No.: US 2015/0157197 A1
(43) Pub. Date: Jun. 11, 2015

(54) ENDOSCOPIC IMAGE OVERLAY

(71) Applicant: Omer Aslam Ilahi, Houston, TX (US)

(72) Inventor: Omer Aslam Ilahi, Houston, TX (US)

(21) Appl. No.: 14/100,660

(22) Filed: Dec. 9, 2013

(51) Int. Cl. A61B 1/317; A61B 1/018; A61B 1/06; A61B 1/04

(52) U.S. Cl. CPC A61B 1/317; A61B 1/04; A61B 1/018; A61B 1/06

(57) ABSTRACT

An apparatus and method for superimposing a reference image on a camera image from an endoscope. The reference image includes indicators, such as angular distance marks. The angular distance marks may be arranged in a circular or arcuate pattern. The angular distance marks may be configured to be continuous or discontinuous. The angular distance marks may be accompanied by corresponding reference information including one or more of clock face numbers, cardinal directions, and numbers of angular degrees.

### BACKGROUND OF THE DISCLOSURE

1. Field of the Disclosure

This disclosure generally relates to the field of surgery and, in particular, endoscopic surgery.

2. Description of the Art

Endoscopes are used for viewing the tissues of a living body from the inside of the body. Endoscopic surgery refers to the use of endoscopes during procedures performed on living tissue. Endoscopes may be specialized based on the area of the body where they are used. An exemplary, but non-limiting, list of endoscopes include: arthroscopes, laparoscopes, and thoracoscopes. Arthroscopes are configured for use in joint surgery, laparoscopes are configured for use inside the abdomen, and thoracoscopes are configured for use in the chest cavity.

Endoscopes are commonly used during minimally invasive surgeries to provide information about the tissues inside the body. A typical endoscope includes rigid and/or flexible telescopic rod lens or rod lenses configured to transmit real-time images of internal body tissues to a camera or cameras (video camera and/or charge coupled device); or the camera(s) may be disposed directly on the end of a rigid and/or flexible telescopic rod. The telescopic rod lens or rod with camera disposed on the end is configured for insertion into the body of an organism. The internal tissues may be illuminated for the camera by a light source configured to transmit light through the telescopic rod or disposed on the end of the rod.

An image of the internal tissues received by the camera is transmitted to a processor that renders the internal image visible on a display such as a monitor or other suitable screen. An operator may use the displayed internal image for navigating through the body, diagnosing a condition, and/or treating a condition.

### Joint Surgery

Minimally invasive joint surgery is often preferred by patients due to the reduced amount of tissue disruption resulting in less pain, less visible scarring, and more rapid recovery times, as compared to open joint surgery. Arthroscopes are specially configured for joint surgical procedures and provide a minimally invasive option to surgeons for diagnosing and correcting conditions in and about many joints, and, in particular, the knee, shoulder, elbow, wrist, ankle, and hip joints. One common type of joint surgery is knee surgery, wherein the surgery is performed on the anterior cruciate ligament (ACL). The ACL is crucial to stabilizing the knee joint during cutting, pivoting, twisting, or jumping activities. This important ligament originates from an attachment to a specific portion of the femur (thigh bone) and inserts onto a specific portion of the tibia (shin bone). ACL reconstruction surgery may be required when the ACL is injured or otherwise not functioning correctly.

### ACL Reconstruction Surgery

ACL reconstruction surgery involves the removal of damaged ACL tissue and reconstruction of the ACL by grafting in replacement tissue consisting of tendon(s) at one or more selected sites in the knee joint. The ACL is commonly considered to be made up of two bundles of tissue, and surgery may involve reconstruction using one (single-bundle) or two (double-bundle) tendon grafts. The locations for bone attachment for both removal and replacement of ACL tissue are known as the femoral attachment and the tibial attachment. To anchor replacement tissue during ACL reconstruction surgery, tunnels at the bone attachments are created and are referred to as the femoral tunnel and the tibial tunnel, respectively. To create the femoral tunnel site for receiving the graft, a pilot hole is usually made at an optimum position for graft placement. The tunnel, which is considerably larger than the pilot hole, is then made around the pilot hole. However, the positioning of the pilot hole and/or tunnel during surgery can be challenging, and is often performed based on an unaided visual assessment of the internal image by the surgeon. Positioning of the site where removal and grafting takes place is a key factor in surgical success and patient recovery. The success of ACL reconstruction surgery is largely determined by a combination of surgical factors, such as graft placement, and post-surgical factors including proper rehabilitation. The success of ACL reconstruction surgery is judged by criteria such as knee range of motion, strength, stability, and functionality, as well as speed of recovery and residual discomfort.

The most common surgical error during ACL surgery is attributed to poor tunnel position—especially that of the femoral tunnel. Poor tunnel position can result in increased stress on the grafted tissue and an increased probability of failure. Although ideal tunnel placement is largely agreed upon in theory; in practice, surgeons fail to agree on the placement of bone tunnels created during ACL reconstruction surgery when visualizing the created tunnels with an arthroscope. See Arthroscopic Agreement Among Surgeons on Anterior Cruciate Ligament Tunnel Placement, Mark O McConkey et al., The American Journal of Sports Medicine, Vol. 40, No. 12 (2012).

### Guided ACL Surgery

Computer assisted orthopedic surgery (CAOS) utilizes a computer to guide the surgeon after reference points in the knee can be identified relative to each other in three dimensional space and using theoretical three dimensional models of normal anatomy. For arthroscopic procedures, where bone landmarks within the joint undergoing surgery are only visible through the arthroscope, CAOS requires attaching rigid reference sensors to bone. For the knee joint, this requires temporarily attaching metal reference sensors to the femur and tibia through incisions that would otherwise not be made were the procedure not to utilize CAOS, thereby increasing the invasiveness of the procedure. Attaching these sensors and using CAOS adds time and expense to endoscopic procedures such as ACL reconstruction, as well as, increases visible scars and postoperative discomfort, which may increase recovery time and even interfere with postoperative rehabilitation, thus potentially compromising ultimate surgical outcome. Also, increasing the invasiveness of the surgical procedure and the length of anesthesia in order to employ CAOS may increase the greater chances of early postoperative infection and anesthetic related complications, respectively. Furthermore, ACL reconstruction surgery using CAOS has been recently found to have a degree of accuracy for tunnel position that is about the same as conventional, non-guided arthroscopic ACL reconstruction surgery. See Computer-Assisted Surgery is Not More Accurate or Precise Than Conventional Arthroscopic ACL Reconstruction, Duncan E. Meuffels et al., Journal of Bone and Joint Surgery, Vol. 94, 1538-45 (2012).

Some attempts have been made to use fluoroscopic guidance during arthroscopic ACL reconstruction surgery to assist with tunnel positioning. However, intraoperative fluoroscopy exposes the patient and surgical team to ionizing radiation, making such techniques undesirable for repeated use. Furthermore, there is the additional cost of intraoperative radiographic equipment and increased operative time, which in-and-of-itself can lead to greater chances of early postoperative complications such as anesthetic related complication and postoperative infection.

### Non-Guided ACL Surgery

Arthroscopic ACL reconstruction surgery requires surgical skill in navigating the knee joint using an arthroscope and determining the exact positions from which to remove and replace tissue. The arthroscopic surgeon may be faced with a narrow space in which to perform the operation and a small field of view generated from the arthroscope. However, the benefits include, but are not limited to, a speedier recovery, less pain, and less scarring, due to the reduction in disturbance of non-ACL tissues during the procedure. Most arthroscopic ACL reconstruction surgeries are currently performed with the surgeon relying on experience and skill while visually gauging the location for tunnel position using just the internal image displayed from the arthroscope. However, the ability of surgeons to consistently determine the exact proper location for creating the femoral tunnel during arthroscopic ACL reconstruction surgery has been recently demonstrated to be rather low, as evidenced by the poor agreement between surgeons regarding tunnel placement when arthroscopically viewing already created tunnels in human knee joints. See Arthroscopic Agreement Among Surgeons on Anterior Cruciate Ligament Tunnel Placement, Mark O McConkey et al., The American Journal of Sports Medicine, Vol. 40, No. 12 (2012).

There is therefore a clear need for an apparatus that enables surgeons to more accurately gauge the position of surgical sites on bodily tissues viewed during endoscopic surgery that does not involve increasing the invasiveness of procedures by requiring insertion of temporary reference monitors or require employing harmful radiation.

### BRIEF SUMMARY OF THE DISCLOSURE

In aspects, the present disclosure is related to the field of endoscopic surgery. Specifically, the present disclosure is related to applying a reference image during endoscopic surgery.

One embodiment includes an apparatus for endoscopic surgical procedures, the apparatus comprising: an electronic display configured to display an image from an endoscope; and a processor configured to superimpose a reference image on the image on the electronic display, the reference image comprising: a plurality of indicators. The indicator may be angular distance indicators. The apparatus may further comprise an endoscope configured to generate the image. The endoscope may be an arthroscope. The reference image may comprise an arcuate shape or a circular shape. The angular distance indicators may be disposed on the circumference of the arcuate shape or the circular shape. The angular distance indicators may comprise clock face numbers.

Another embodiment includes a method of providing indicators during endoscopic surgery, the method comprising: superimposing a reference image on an electronic display of an image generated by an endoscope, wherein the reference image comprises a plurality of indicators. The indicators may be angular distance indicators. The reference image may comprise an arcuate shape or a circular shape. The angular distance indicators may be disposed on the circumference of the arcuate shape or the circular shape. The angular distance indicators may comprise clock face numbers.

Another embodiment includes a non-transitory computer-readable medium product, the medium containing instructions thereon that, when executed by a processor, executes a method, the method comprising: superimposing a reference image on an electronic display, wherein the electronic display is configured to receive an image from an endoscope and wherein the reference image comprises a plurality of indicators. The indicators may be angular distance indicators. The medium may comprise at least one of: i) a ROM, ii) an EPROM, iii) an EEPROM, iv) a flash memory, v) an optical disk, and vi) a hard drive.

Examples of the more important features of the disclosure have been summarized rather broadly in order that the detailed description thereof that follows may be better understood and in order that the contributions they represent to the art may be appreciated. There are, of course, additional features of the disclosure that will be described hereinafter and which will form the subject of the claims appended hereto.

### BRIEF DESCRIPTION OF THE DRAWINGS

For a detailed understanding, reference should be made to the following detailed description of the embodiments, taken in conjunction with the accompanying drawings, in which like elements have been given like numerals, wherein:

FIG. 1 is a schematic of an endoscope attached to a display according to one embodiment of the present disclosure;

FIG. 2 is an end view of a left human femur with a reference image superimposed according to one embodiment of the present disclosure;

FIG. 3A is an exemplary reference image with a circular clock face pattern according to one embodiment of the present disclosure;

FIG. 3B is an exemplary reference image with a circular compass pattern according to one embodiment of the present disclosure;

FIG. 3C is an exemplary reference image with circular angular degree pattern according to one embodiment of the present disclosure;

FIG. 3D is an exemplary reference image with an arcuate clock face pattern according to one embodiment of the present disclosure;

FIG. 3E is an exemplary reference image with an arcuate, discontinuous clock face pattern according to one embodiment of the present disclosure;

FIG. 3F is an exemplary reference image with an arcuate, discontinuous pattern with quadrants according to one embodiment of the present disclosure; and

FIG. 4 is a flow chart of a method of superimposing a reference image on an endoscope image according to one embodiment of the present disclosure.

### DETAILED DESCRIPTION OF THE DISCLOSURE

In aspects, the present invention is related to a reference image for surgery. Specifically, the present invention is related to generating a reference image that is superimposed on an image from an endoscope. The present invention is susceptible to embodiments of different forms. There are shown in the drawings, and herein will be described in detail, specific embodiments with the understanding that the present invention is to be considered an exemplification of the principles and is not intended to limit the present invention to that illustrated and described herein.

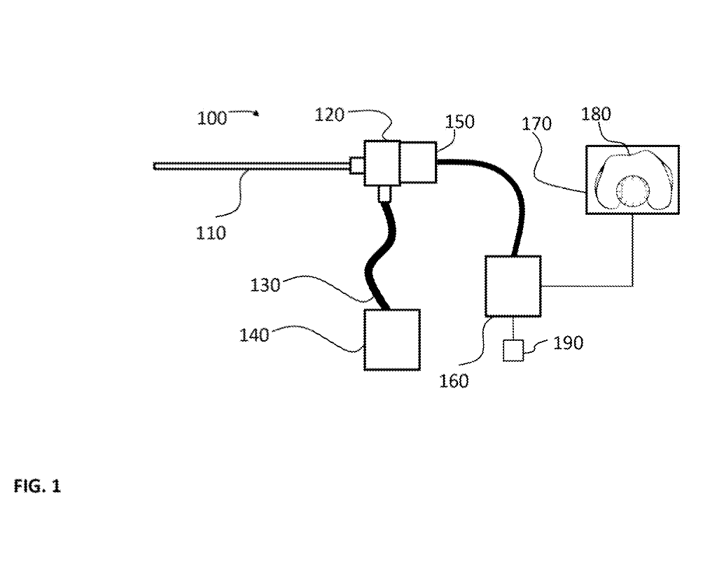

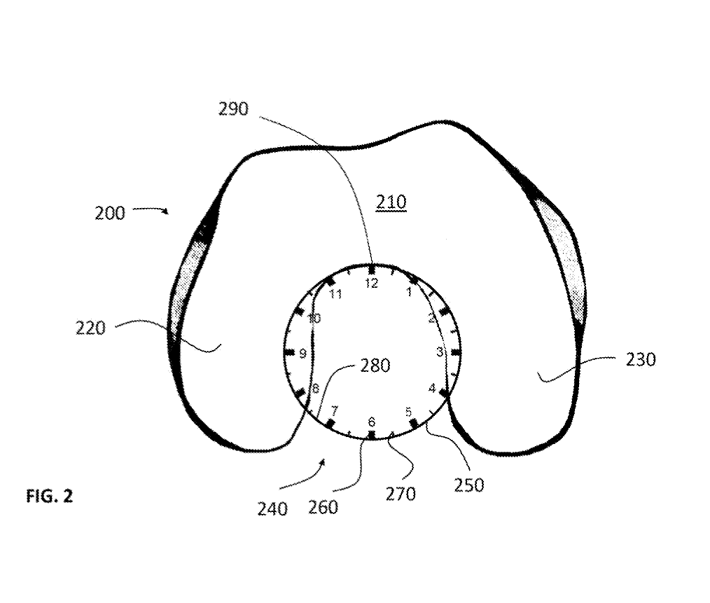

FIG. 1 shows an exemplary endoscope system 100. The system includes a telescopic lens 110 configured to communicate visible light along its length. The telescopic lens 110 is disposed on a housing 120. The housing 120 is configured to receive a light cable 130 from a light source 140. The housing 120 is also configured to receive a camera 150. In some embodiments, the camera 150 is configured to be disposed on the end of a telescopic rod (not shown). An image captured by the camera 150 may be transmitted to a processor 160 which converts the image into signals (electronic image) that may be viewed on a display 170. The displayed image 180 is shown within an electronic display 170. The processor 160 may include a memory (not shown) with instructions for adding a reference image 250 (see FIG. 2) to an image captured from the camera 150. In some embodiments, the instructions for generating the reference image 250 may be stored on a separate memory 190 that is in communication with the processor 160.

The memory in processor 160 and optional separate memory 190 are non-transitory computer-readable media that may include any standard non-transitory computer information storage device, such as a ROM, USB drive, memory stick, hard disk, removable RAM, EPROMs, EAROMs, EEPROM, flash memories, and optical disks or other commonly used memory storage system known to one of ordinary skill in the art including Internet based or cloud storage.

FIG. 2 shows a display image 200 of an end view of a left human femur 210. Display image 200 may be used as displayed image 180 in FIG. 1. The display image 200 shows the medial condyle 220, lateral condyle 230, and intercondylar notch 240 of the femur 210. The reference image 250 may be superimposed over the image of the femur 210. The reference image 250 is configured to provide indicators, such as angular distance reference points to aid a user in estimating the angular position of one or more surgical sites relative to a known reference point on the image 200. The reference image 250 may include a plurality of angular distance indicators or markers 260, 270. The angular distance markers 260, 270 may include major increment markers 260, minor increment markers 270, or both. As shown, the reference image 250 is arranged as a clock face with major increment markers 260 at hourly positions (every 30 degrees) and minor increment markers 270 at half-hourly positions (every 30 degrees offset by 15 degrees from the hourly positions). It is contemplated that any range of increment sizes may be used in the reference image 250. The reference image 250 also includes an optional circular ring 280 aligned with the angular distance markers 260, 270. The ring 280 may be used to guide magnification adjustment of the reference image 250, so that key reference points and surgical sites are aligned with the angular distance markers 260, 270.

In operation, the user, usually a surgeon, may align one of the major increment markers 260, such as the 12 o'clock position with a known reference point on the femur 210 as is understood and determined by the surgeon's expertise. For example, in arthroscopic ACL reconstruction surgery, the 12 o'clock position may be aligned with the top center position 290 of the femoral intercondylar notch 240. The top (or “roof) of the femoral intercondylar notch 240 is variable from person to person; however, the top is generally small in angular size relative to the length of the walls of the of the femoral intercondylar notch 240. The alignment between the known reference point and the 12 o'clock position may be performed by the surgeon rotating the camera 150 (or the entire endoscope) until the known reference point is aligned with the 12 o'clock position. The angular location of the surgical site may then be determined with greater accuracy as the surgeon is able to visually determine the location of the surgical site relative to the angular distance markers 260, 270. Alignment with the 12 o'clock position is exemplary and illustrative only, as a person of ordinary skill in the art would understand that other reference points may be used for alignment. One example of an alternative alignment is centering the top half of the reference image 250 on the baseline of the femoral intercondylar notch 240. In one embodiment, the baseline may be estimated as a line between the lowest point of the medial condyle 220 and the lowest point of the lateral condyle 230. In another embodiment, the baseline may be estimated based on the superior aspect of the tibia when the knee is in the bent position for ACL reconstruction surgery. While the above description is directed to the use of the superimposed reference image in the context of endoscopic ACL reconstruction surgery, this is exemplary and illustrative only, as other forms of endoscopic procedures may use the superimposed reference image, including, but not limited to, other knee surgeries, endoscopic exploratory surgeries, laparoscopic procedures, and thoracoscopic procedures.

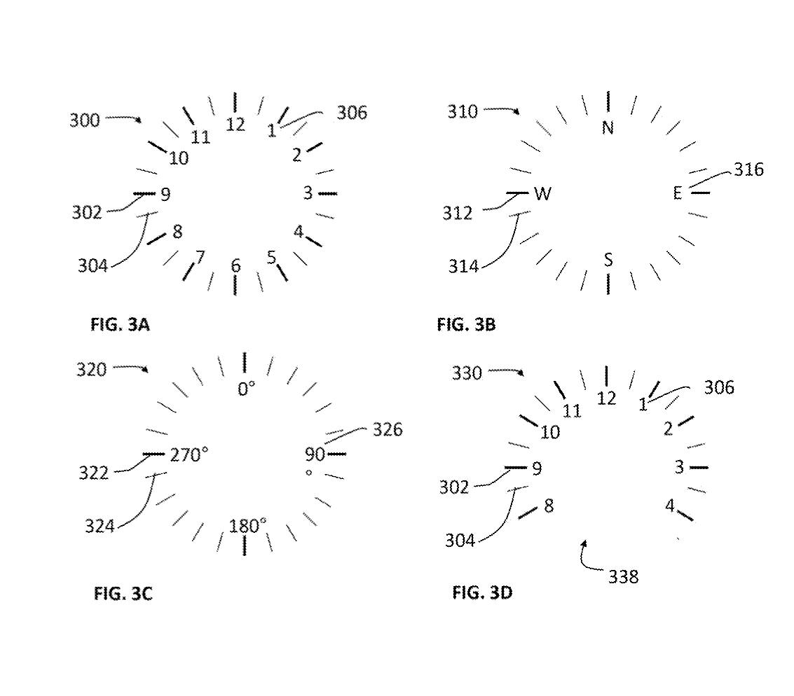

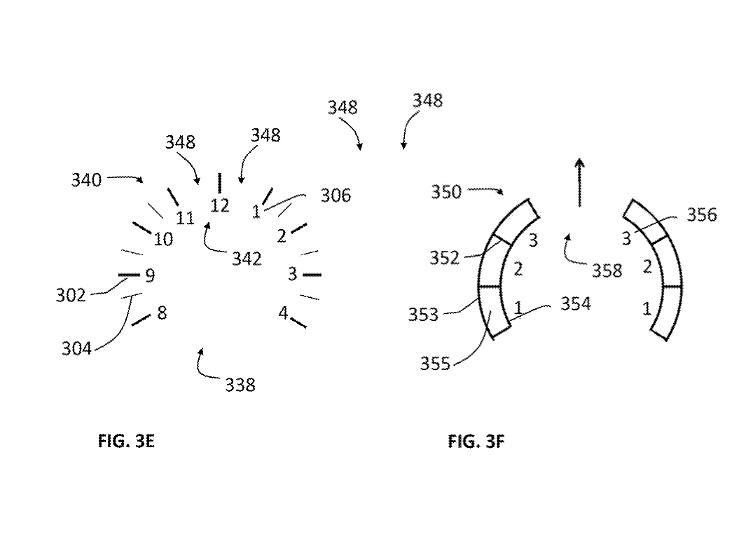

FIG. 3A shows an exemplary reference image 300 with a full clock face where a plurality of indicators, such as angular distance markers 302, 304 are configured in a circular pattern with alternating major increments 302 and minor increments 304. The major increments 302 are identified by hour numerals 306. In some embodiments, the clock face may include more or fewer hour numerals than the twelve shown in FIG. 3A.

FIG. 3B shows an exemplary reference image 310 with a full compass face where a plurality of indicators, such as angular distance markers 312, 314 are configured in a circular pattern with major increments 312 and minor increments 314. Each of the major increments 312 are associated with a cardinal direction indicator 316. Each of the minor increments 314 is disposed such that each of the angular distance markers 312, 314 is separated by 15 degrees from the adjacent angular distance markers. The use of 15 degrees for separation is illustrative and exemplary only, as size of separation, both uniform and nonuniform, is contemplated.

FIG. 3C shows an exemplary reference image 320 with angular degree numbers where a plurality of indicators, such as angular distance markers 322, 324 are configured in a circular pattern with major increments 322 and minor increments 324. Each of the major increments 322 represents an angular quarter of the circle and is accompanied by a degree numeral 326. Each of the angular distance markers 322, 324 is separated from an adjacent marker by 15 degrees. The 15 degree separation distance is exemplary and illustrative only, as any angular separation distance may be used.

FIG. 3D shows an exemplary reference image 330 with the clock face numbers 306 configured in an arcuate shape. The reference image 330 includes a gap 338 wherein no indicator or angular distance markers 302, 304 or clock face numbers 306 are shown. The gap 338 may be selected to provide a section of the reference image 330 that is reserved for another superimposed image or to allow clearer viewing of the underlying image. As shown, the arcuate shape may include a partial clock face that ranges from about 8 o'clock to about 4 o'clock (moving clockwise), though the starting point, ending point, and size of the partial clock face range may be configured by the user. The arcuate shape may include partial or incomplete circular shapes, including half circles and three-quarter circles. The arcuate shape is shown as a partial circle configuration of angular distance markers 302, 304; however, other arcuate shapes are contemplated, including partial ovoid and partial elliptical shapes. In some embodiments, the arcuate shape may include any curve formed by a plurality of angular distance markers 302, 304. The angular distance markers 302, 304 may be uniform or non-uniform.

FIG. 3E shows a reference image 340 with an arcuate, discontinuous configuration of the indicators, such as angular distance markers 302, 304 and hour numbers 306. The reference image 340 includes continuous angular distance markers from 8 o'clock to 11 o'clock and from 1 o'clock to 4 o'clock with a reference point marker at 12 o'clock, which are separated by the gap 338 at the bottom and by gaps 348 at the top. The top gaps 348 are separated by a major increment marker 306 that also serves as a reference point marker 342 and may be used to align the image viewed by the endoscope system 100.

FIG. 3F shows a reference image 350 with an arcuate, discontinuous configuration of the indicators, such as angular distance markers 352. The reference image 350 includes outer arcuate sections 353 and inner arcuate sections 354 which border the angular distance markers 352 to form quadrants 355. The reference image 350 may include quadrant identifiers 356. The reference image 350 may also include a reference point marker 358 that may be aligned with a reference point on the patient, such as the center of the femoral intercondylar notch 240. As shown, each of the quadrants 355 covers 30 angular degrees; however, this dimension is exemplary and illustrative only, as the quadrants 355 may be formed to have any angular size, and the quadrants 355 do not have to be of identical size. The dimensions of the quadrants 355 may be adjusted based on preference of the user or due to the type of endoscopic surgery being performed.

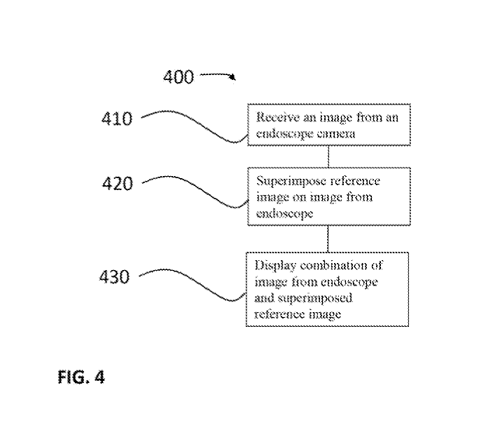

FIG. 4 shows a flow chart for a method 400 of applying an image overlay for an endoscope image according to one embodiment or more embodiments of the present disclosure. In step 410, an internal image is received by the camera 150 of the endoscope system 100. In step 420, a reference image 250 is superimposed on the internal image received by the camera 150. In step 430, the combination of the camera image and the reference image 250 are displayed on a monitor 170 or other suitable display, including but not limited to Google™ glass or other eyewear. The reference image 250 includes a plurality of indicators, such as angular distance markers 302, 304, which may include suitable marks to indicate angular distance positions from a reference point selected by the user, including, but not limited to, hash marks, line segments, and dots. The reference image 250 may comprise, but is not limited to, a suitable reference image such as reference images 300, 310, 320, 330, 340, 350.

While the invention has been described with reference to exemplary embodiments, it will be understood that various changes may be made and equivalents may be substituted for elements thereof without departing from the scope of the invention. In addition, many modifications will be appreciated to adapt a particular instrument, situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. An apparatus for endoscopic surgical procedures, the apparatus comprising:
an electronic display configured to display an image from an endoscope; and
a processor configured to superimpose a reference image on the image on the electronic display, the reference image comprising:
a plurality of indicators.

2. The apparatus of claim 1, wherein the indicators are angular distance indicators.

3. The apparatus of claim 2, further comprising:
an endoscope configured to generate the image.

4. The apparatus of claim 3, wherein the endoscope is an arthroscope.

5. The apparatus of claim 2, wherein the reference image comprises an arcuate shape.

6. The apparatus of claim 5, wherein the plurality of angular distance indicators are disposed on a circumference of the arcuate shape.

7. The apparatus of claim 2, wherein the reference image comprises a circular shape.

8. The apparatus of claim 7, wherein the plurality of angular distance indicators are disposed on a circumference of the circular shape.

9. The apparatus of claim 2, wherein the angular distance indicators comprise clock face numbers.

10. A method of providing indicators during endoscopic surgery, the method comprising:
superimposing a reference image on an electronic display of an image generated by an endoscope, wherein the reference image comprises a plurality of indicators.

11. The method of claim 10, wherein the indicators are angular distance indicators.

12. The method of claim 11, wherein the reference image comprises an arcuate shape.

13. The method of claim 12, wherein the plurality of angular distance indicators are disposed on a circumference of the arcuate shape.

14. The method of claim 11, wherein the reference image comprises a circular shape.

15. The method of claim 14, wherein the plurality of angular distance indicators are disposed on a circumference of the circular shape.

16. The method of claim 11, wherein the angular distance indicators comprise clock face numbers.

17. The method of claim 11, wherein the endoscope is an arthroscope.

18. A non-transitory computer-readable medium product, the medium containing instructions thereon that, when executed by a processor, executes a method, the method comprising:
superimposing a reference image on an electronic display, wherein the electronic display is configured to receive an image from an endoscope and wherein the reference image comprises a plurality of indicators.

19. The product of claim 18, wherein the indicators are angular distance indicators.

20. The non-transitory computer-readable medium product of claim 18, wherein the medium comprises at least one of: i) a ROM, ii) an EPROM, iii) an EEPROM, iv) a flash memory, v) an optical disk, and vi) a hard drive.
